(19) United States
(12) Patent Application Publication
(10) Pub. No.: US 2024/0293523 A1
(43) Pub. Date: Sep. 5, 2024

(54) VACCINE METHOD AND COMPOSITION FOR BACTERIAL DISEASES IN INVERTEBRATES

(71) Applicant: Dalan Animal Health, Inc., Los Angeles, CA (US)

(72) Inventors: Annette Kleiser, Los Angeles, CA (US); Dalial Freitak, Los Angeles, CA (US)

(21) Appl. No.: 18/549,569

(22) PCT Filed: Mar. 11, 2022
(86) PCT No.: PCT/US22/20066

§ 371 Date: Sep. 7, 2023

Related U.S. Application Data
(60) Provisional application No. 63/160,535, filed on Mar. 12, 2021.

(51) Int. Cl. A61K 39/09; A61K 39/00; A61K 39/02; A61P 37/04

(52) U.S. Cl. CPC A61K 39/09; A61K 39/0208; A61P 37/04; A61K 2039/521; A61K 2039/542; A61K 2039/552; A61K 2039/58

(57) ABSTRACT

The disclosure provides compositions and methods for vaccinating invertebrates and invertebrate populations from diseases. The disclosure further provides compositions and methods for prophylactically immunizing honeybee hive to protect from infection with Foulbrood disease. In embodiments, the disclosure further provides compositions and methods for prophylactically immunizing honeybee hive to protect from infection with European foulbrood or American foulbrood caused by Melissococcus plutonius using a non¬disease causing bacterium.

### CROSS-REFERENCE TO RELATED APPLICATION

This application claims the benefit under 35 U.S.C. § 119(e) of U.S. Provisional Application Ser. No. 63/160,535, filed Mar. 12, 2021, the contents of which are incorporated herein by reference.

### BACKGROUND

Invertebrates such as honeybees, shrimp, and edible insects for food and feed production play a critical role in our food supply chain. Diseases caused by bacteria, viruses and fungi can infect these animals at different stages of their life cycle and represent a limiting factor for growth in these industries. Diseases are responsible for up to 40% capacity lost each year.

Honeybees are used commercially as essential species providing pollination services, as well as honey production. In the United States, commercial beekeepers operate approximately 2.8 million beehives, and honeybees are responsible for pollinating more than one third of all crops including crops such as nuts, berries, fruit, and flowering vegetables. In addition, honeybees also pollinate a variety of wild flowers and, therefore, contribute to the biodiversity of many ecosystems.

Unfortunately, in recent years honeybee colonies have suffered severe declines and colony losses. For example, in the year spanning April 2018-April 2019, over 40% of the 2.8 million commercially operated beehives in the US died; these losses not only result in millions of dollars in lost revenue to U.S. beekeepers, and revenue losses to orchard owners and other farmers due to the lack of pollinating insects, but also impact job security with 22,000 jobs depending on the beekeeping industry in the U.S., our food security and international trade of agricultural products.

Chief among the causes of the loss of honeybee colonies are pests and pathogens, including parasites, and viral and bacterial infections.

Particularly prevalent bacterial diseases are American Foulbrood AFB and European Foulbrood (EFB). American Foulbrood is caused by the bacterium Paenibacillus larvae and European Foulbrood (EFB) is caused by the bacterium Melissococcus plutonius. The diseases weaken the hive as they kill the brood and cause farmers and beekeepers significant losses. In contrast to AFB whose spores are resistant to antibiotic treatment, EFB can be managed by application of antibiotic Terramycin. However, low level of bacteria can remain in the hive and cause reoccurring disease for years. Furthermore, contamination of honey and the environment with antibiotics and the increase in antibiotic resistance is of growing concern to the industry and regulator. Hence, its is suggested to burn EFB infected hives similarly to AFB infected ones in many countries. Unfortunately, there are currently no safe and effective prophylactic solutions available to protect hives from bacterial diseases including EFB and AFB.

Likewise, antibiotics are unsafe for use to treat or prevent bacterial diseases in other invertebrates used in food production. The development of safe and sustainable solutions to protect these critical life stock industries is of critical importance for the food security.

Vaccination is most effective strategy for disease prevention and containment in many life stock species.

Thus, there is a need in the art for safe, effective measures for the control and prevention of foulbrood disease and other related invertebrate and insect disease. This disclosure satisfies this need and provides related advantages as well.

### SUMMARY

The disclosure relates to a novel vaccine formulation and methods for the prevention of a bacterial disease in bees, honeybees, insects and other invertebrates including but not limiting to shrimp, grasshoppers and mealworms and other pollinators whereby the maternal invertebrate or insect (e.g. honeybee queen) is vaccinated with a dead bacteria that is not the disease causing bacteria. The disclosure also relates to a novel vaccine formulation and methods for the prevention of a bacterial disease in bees, honeybees, insects and other invertebrates including but not limiting to shrimp, grasshoppers and mealworms and other pollinators whereby the maternal invertebrate or insect (e.g. honeybee queen) is vaccinated with a dead non-disease causing bacteria Enterococcus, e.g., Melissococcus sp., e.g., Melissococcus plutonius. Vaccination of queen bees with dead Melissococcus plutonius will protect against brood diseases, e.g., caused by Enterococcus, e.g., Melissococcus sp. or Melissococcus plutonius and other disease causing bacteria, e.g. Paenibacillus sp., including, but not limited to Paenibacillus alvei (PA), Paenibacillus dentritiformis (PD), or Paenibacillus larvae (PL). In addition, vaccination with Paenibacillus sp., including, but not limited to Paenibacillus alvei (PA), Paenibacillus dentritiformis (PD), or Paenibacillus larvae (PL) will protect against brood diseases caused by disease pathogens other than a disease caused by a Paenibacillus sp., including, but not limited to Paenibacillus alvei (PA), Paenibacillus dentritiformis (PD), or Paenibacillus larvae (PL) or a diseases caused by Melissococcus plutonius.

In one aspect, the bacterial species in the vaccine is non-disease causing bacterial species. In one aspect, the vaccine composition comprise a non-disease causing bacterial species and comprises whole cells or cell wall fragments of at least one dead non-disease causing bacterial species. In another aspect, the vaccine composition can comprise dead Melissococcus plutonius and dead Paenibacillus sp. In one embodiment, the composition also can contain, for example, invertebrate, insect, bee or honeybee feed.

Further, the disclosure relates to generally immunizing invertebrates to protect against disease by using non-disease causing bacteria. In further embodiments, the honeybee as disclosed herein can be substituted with any other insect or invertebrate. In some embodiments, the insect is a social insect.

The disclosure also provides a vaccine composition comprising Melissococcus plutonius bacteria that is useful to immunize invertebrates to protect against disease by non-Melissococcus plutonius bacterial disease causing bacteria. In one aspect, the vaccine composition comprise a non-disease causing bacterial species and comprises whole cells or cell wall fragments. The composition also can contain, for example, invertebrate, insect or honeybee feed. In further embodiments, the honeybee as disclosed herein can be substituted with any other insect or invertebrate. In some embodiments, the insect is a social insect.

In contrast to what had been known in the art, Applicant describes immunization by applying a stimulus from a non-disease pathogen to prophylactically immunize the invertebrates, among them insects including bees against a certain disease such as EFB or AFB. Specific match between the disease pathogen and the vaccine strain is not required.

Thus, in one aspect, this disclosure provides a method of protecting invertebrates from disease by applying or administering an immunization stimulus derived from a pathogen other than the disease pathogen to provide broad-spectrum generalized protection. As an example, this disclosure provides a method of protecting invertebrates from disease by applying or administering an immunization stimulus derived from a dead Melissococcus plutonius to protect against a disease caused by a pathogen other than a Melissococcus plutonius pathogen to provide broad-spectrum generalized protection.

In other embodiments, the disclosure provides a method for preventing EFB in a population of invertebrates, insects, bees or honeybees. The method can include administering to the worker bees and the queen bee or similar invertebrate or insect populations a vaccine formulation comprising dead Paenibacillus such as PL, PD, PA, or a combination thereof with for example Melissococcus sp. such as Melissococcus plutonius.

In one aspect and in the case of treating or preventing EFB, the non-disease causing bacterial species for the composition formulation is a species of the genus Paenibacillus, such as, Paenibacillus alvei, Paenibacillus larvae or Paenibacillus dentritiformis, or a combination thereof. In a further aspect, the one or more, two or more, three or more, or four or more dead Paenibacillus species is included in the composition, wherein the species is one or more selected from the group consisting of Paenibacillus alvei, Paenibacillus larvae, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, Paenibacillus xylanisolvens, or a combination thereof. In a further aspect, the dead Paenibacillus species in the composition are Paenibacillus alvei, Paenibacillus larvae, and Paenibacillus dentritiformis.

In the case of AFB, non-limiting examples of the non-disease causing bacterial species for the composition formulation is a species of the genus Melissococcus, such as, Melissococcus plutonius. In a specific embodiment, provided herein is a composition, comprising, or consisting essentially of, or yet further consisting of, whole cells or cell wall fragments of at least one dead non-disease causing or disease causing bacterial species of a bacterial genus and a carrier. In one aspect, the composition comprises, or consisting essentially of, or yet further consisting of, whole cells or cell wall fragments of at least two or more, or three or more, of four or more dead, non-disease causing or disease causing bacterial species of a bacterial genus and a carrier. A carrier can be a solid or a liquid carrier and can include preservatives, insect nutrients, or other coloring agents as necessary. In one specific embodiment, the carrier is an invertebrate or insect food, such as a queen wafer or sugar feed.

In a specific embodiment, provided herein is a composition, comprising, or consisting essentially of, or yet further consisting of, whole cells or cell wall fragments of at least one dead, non-disease causing and optionally, a disease causing bacterial species of a bacterial genus and a carrier. In one aspect, the composition comprises, or consisting essentially of, or yet further consisting of, whole cells or cell wall fragments of at least two or more, or three or more, of four or more dead, non-disease causing or disease causing bacterial species of a bacterial genus and a carrier. A carrier can be a solid or a liquid carrier and can include preservatives, insect nutrients, or other coloring agents as necessary. In one specific embodiment, the carrier is an invertebrate or insect food, such as a queen wafer or sugar feed. The composition also can contain, for example, invertebrate, insect, bee or honeybee feed.

In an exemplary embodiment, provided herein is a composition containing dead Paenibacillus sp. such as PL optionally in combination with other dead Paenibacillus sp. or Melissococcus sp. for the protection against infection by Melissococcus plutonius (MP).

The composition also can contain, for example, invertebrate, insect or honeybee feed. In another exemplary embodiment, provided herein is a composition containing dead Melissococcus such as Melissococcus plutonius optionally in combination with dead Paenibacillus sp. for the protection against infection by Paenibacillus sp. The composition also can contain, for example, invertebrate, insect, bee or honeybee feed, and a carrier. A carrier can be a solid or a liquid carrier and can include preservatives, insect nutrients, or other coloring agents as necessary.

In one aspect, the whole cells or cell wall fragments of at least one dead, non-disease causing bacterial species of the bacterial genus in the composition are present in a total amount per dose of between about 1.5×10⁷ to about 1.5×10¹¹ antigen units, or from about 1.5×10⁸ to about 1.5×10¹¹ antigen units, or from about 1.5×10⁸ to about 3×10¹⁰ antigen units, or from about 1.5×10⁸ to about 1.5×10⁹ antigen units, or at least 1.5×10⁷ antigen units, or at least about 1.5×10⁸ antigen units, or at least about 1.5×10⁹ antigen units, or at least about 1.5×10¹⁰ antigen units.

Also provided herein is a method of immunizing an invertebrate, among these an insect (including but not limiting to, a social insect such as a honeybee larvae) against disease, the method comprising, or alternatively consisting essentially of, or yet further consisting essentially of, administering an effective amount of the composition as described herein to a nurse bees or the honeybee queen or similar invertebrates or insects in those populations, thereby immunizing the invertebrate, insect or queen bee, and the progeny thereof, e.g., honeybee larvae produced by the queen bee. In one aspect, the disease is caused by a species of Melissococcus and in another aspect the disease is caused by Paenibacillus.

The method is useful to protect larvae from European Foulbrood (EFB), and is caused by Melissococcus plutonius and to provide a population of larvae that has been immunized against American Foulbrood from the queen bee that has ingested the vaccine composition.

Further provided herein is a method for preventing European foulbrood (EFB) in a population of honeybees comprising, or alternatively consisting essentially of, or yet further consisting of administering to worker bees and the queen bee, a composition as described herein, wherein in one aspect the composition comprises, or consists essentially of, or yet consisting of dead non-disease whole or fragments from the species of Paenibacillus. In another aspect, the dead non-disease species of Paenibacillus are selected from the group consisting of Paenibacillus larvae, Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, Paenibacillus xylanisolvens, or a combination thereof. In another aspect, the composition comprises one or both of dead non-disease species of Paenibacillus are dead Paenibacillus alvei and Paenibacillus dentritiformis, or a combination of both in combination with other Paenibacillus, Melissococcus or other inactivated cells or fragments of bacteria of the order Bacillus.

In one aspect of this method, the administering comprises feeding the worker bees and the queen bee, wherein the amount administered per dose a vaccine comprising between about 1.5×10⁵ to about 1.5×10¹¹ antigen units, or from about 1.5×10⁷ to about 1.5×10¹¹ antigen units, or from about 1.5×10⁸ to about 1.5×10¹¹ antigen units, or from about 1.5×10⁵, or from about 1.5×10⁶, or from about 1.5×10⁷, or from about 1.5×10⁸ to about 1.5×10¹⁰ antigen units, or from about 1.5×10⁸ to about 1.5×10⁹ antigen units, or at least 1.5×10⁷ antigen units, or at least about 1.5×10⁸ antigen units, or at least about 1.5×10⁹ antigen units, or at least about 1.5×10¹⁰ antigen units, or any range(s) or number(s) therebetween, of the at least one dead non-disease species of Paenibacillus or dead, species of Melissococcus or fragments, or each thereof.

Further provided is a method for preparing an insect vaccine comprising isolating from at least one, or at least two, or at least three, or at least four or more dead, non-disease causing bacterial species of a bacterial genus, whole cells or cell wall fragments from the at least one dead, non-disease causing bacterial species of the bacterial genus or alternatively, a dead, Melissococcus bacterial species. In one embodiment, the method further comprises admixing the isolated antigen units with an insect food or carrier, such as a queen bee wafer. In one aspect, the non-disease causing bacterial species is a species of the genus Paenibacillus, such as, Paenibacillus larvae, Paenibacillus alvei or Paenibacillus dentritiformis, or a combination thereof. In another aspect, the dead Paenibacillus species is selected from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, Paenibacillus xylanisolvens, or a combination thereof. In another aspect, the dead Paenibacillus species are Paenibacillus alvei and Paenibacillus dentritiformis. In one aspect, the dead, species of Melissococcus, e.g., Melissococcus plutonius. Additionally or alternatively, the non-disease causing bacterial species is a species of other bacilli species.

In one aspect, the whole cells or cell wall fragments of at least one, or at least two, or at least three, or at least four or more of dead, non-disease causing bacterial species of a bacterial genus per does is provided between about 1.5×10⁵, 1.5×10⁶, or 1.5×10⁷ to about 1.5×10¹¹ antigen units, or from about 1.5×10⁸ to about 1.5×10¹¹ antigen units, or from about 1.5×10⁸ to about 1.5×10¹⁰ antigen units, or from about 1.5×10⁸ to about 1.5×10⁹ antigen units, or at least 1.5×10⁷ antigen units, or at least about 1.5×10⁸ antigen units, or at least about 1.5×10⁹ antigen units, or at least about 1.5×10¹⁰ antigen units, or any range(s) or number(s) therebetween, of the dead non-disease species of Paenibacillus, or dead, inactivated or attentuated Melissococcus or fragments of each thereof.

Also provided herein are the vaccine compositions prepared by the methods as described herein as well as the vaccination of the brood by ingestion of the vaccine by the material invertebrate, among these insect, social insect or honeybee as described herein. The compositions can be provided, but not limited to, in a kit, comprising the composition as described herein and instructions for use.

Other features, objects and advantages of the disclosure will be apparent from the detailed description which follows.

### BRIEF DESCRIPTION OF THE DRAWINGS

FIGS. 1A to 1C illustrate the mechanism of vaccination against AFB and EFB in honeybees. 1) Vaccination: Worker bees are fed a composition as disclosed herein. Conventional queen feed (queen candy) comprising the composition (the oral vaccine) is taken up by the worker or nurse bees and added to royal jelly in the mandibular glands. The queen bee or the queen larvae is fed the vaccine containing royal jelly by the worker and nurse bees. 2) The vaccine is stored in the fat bodies of the larvae and the queen bee and, once the queen is fully developed, transported to the ovaries of the queen bee. The transfer is mediated by the egg yolk protein Vitellogenin. 3) Efficacy: Queen vaccination with dead Paenibacillus sp and other bacilli species results in more resistant larvae then infected with the pathogen (Red bars, vaccine: XXX larval survival vs. placebo: XXX larva survival, χ2=XXX, p=XXX). No negative effect on larval survival under control conditions with no infection (Black bars, vaccine: XXX larval survival vs. placebo: XXX larva survival, χ2=XXX, p=XXX). FIG. 1C shows that vaccinated larvae are significantly more resistant to disease (p<0.04), an important indicator for even greater efficacy in the field. Vaccination has no negative impact on larval fitness-both groups show equal background mortality. N=9 hives (2 vaccinated, 7 unvaccinated controls).

FIG. 2 illustrates a method for vaccine production.

FIG. 3 shows ross-protection provided by AFB vaccine (1.5×10⁸) against the infection with European FB (spores). Sample sizes in different treatments are shown on the corresponding bars.

FIG. 4 shows cross-protection provided by EFB vaccine (2.14×10⁸) against the infection with AFB (spores). Sample sizes in different treatments are shown on the corresponding bars.

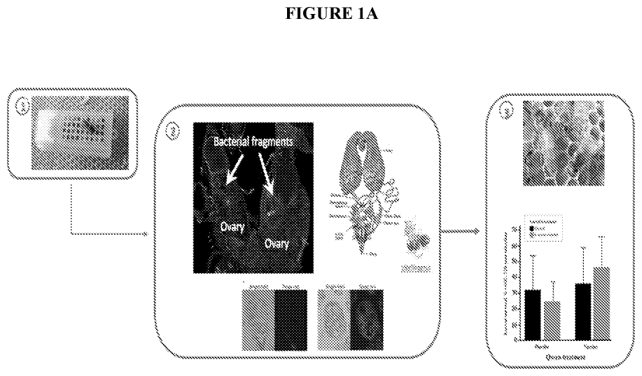

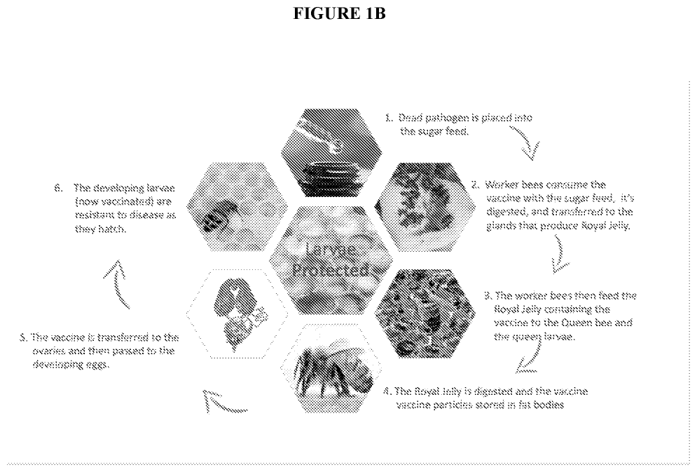

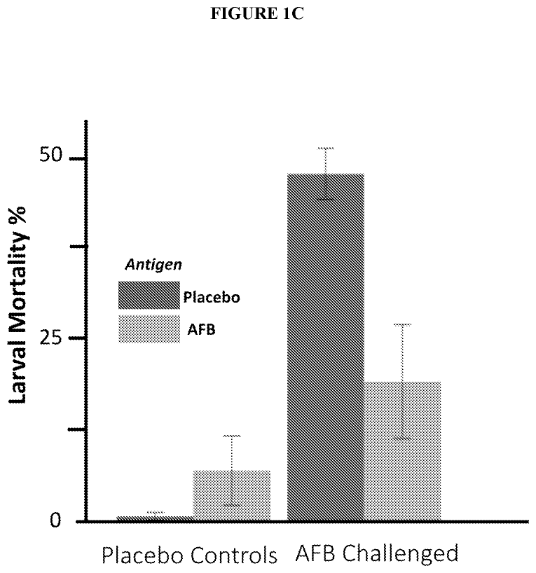

### DETAILED DESCRIPTION

### Definitions

As used herein and in the appended claims, singular articles such as “a” and “an” and “the” and similar referents in the context of describing the elements are to be construed to cover both the singular and the plural, unless otherwise indicated herein or clearly contradicted by context.

As used herein, “about” is understood by persons of ordinary skill in the art and may vary to some extent depending upon the context in which it is used. If there are uses of the term which are not clear to persons of ordinary skill in the art given the context in which the term “about” is used, “about” will mean up to plus or minus 10% of the particular term.

As will be understood by one skilled in the art, for any and all purposes, all ranges disclosed herein also encompass any and all possible subranges and combinations of subranges thereof. Furthermore, as will be understood by one skilled in the art, a range includes each individual member.

The term “exemplary” as used herein refers to “serving as an example, instance, or illustration,” and not “preferred” or “advantageous over other embodiments.”

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

Unless defined otherwise, technical and scientific terms used herein have the same meaning as commonly understood by a person of ordinary skill in the art. In particular, this disclosure utilizes routine techniques in the field of honeybee husbandry.

The term “European foulbrood” or “European Foulbrood disease” or “EFB” as used herein, refers to a fatal bacterial disease of honeybee brood caused by bacterium Melissococcus plutonius. Since Melissococcus plutonius causes European foulbrood disease in honeybees, in some embodiments, Melissococcus plutonius is referred to herein as a “disease-causing” bacterium or a “disease species.”

The term “American foulbrood” or “American Foulbrood disease” or “AFB” as used herein, refers to a fatal bacterial disease of honeybee brood caused by bacterium Paenibacillus larvae. Since Paenibacillus larvae causes American Foulbrood disease in honeybees, in some embodiments, Paenibacillus larvae is referred to herein as a “disease-causing” bacterium or a “disease species.”

The terms “non-disease causing” pathogen or “non-disease species” as used herein refer to species of bacteria which do not cause the disease being targeted. For example, with respect to European foulbrood disease which is caused by Melissococcus plutonius, exemplary non-disease species, which may be found for example in the environment, but not on humans or in human wounds may include e.g., Paenibacillus larvae, Paenibacillus alvei, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, Paenibacillus xylanisolvens, or other bacterial species in the order of Bacillus.

The term “honey bee” as used herein refers to is any bee which is a member of the genus Apis, primarily distinguished by the production and storage of honey and the construction of perennial, colonial nests from wax. For example, two species of honey bees, namely A. mellifera or A. cerana indica, are often maintained by beekeepers. Honey bees include but are not limited to Apis andreniformis and Apis florea in subgenus Micrapis, Apis dorsata in subgenus Megapis, and Apis cerana, Apis koschevnikovi, Apis mellifera and Apis nigrocincta in subgenus Apis.

In some embodiments, a honeybee as disclosed herein can be substituted with any other insect or invertebrate. In some embodiments, the insect is a social insect.

Accordingly, as used herein, the term “insect” refers to pancrustacean hexapod invertebrates and the largest group within the arthropod phylum. In some embodiments, an insect refers to any member of a large group of invertebrate animals characterized in the adult state by division of the body into head, thorax, and abdomen, three pairs of legs, and often (but not always) two pairs of membranous wings. This definition also includes non-adult insect states include egg, larva and pupa.

The term “social insect” refers to an insect showing eusociality, i.e., the highest level of organization of sociality defined by one or more the following characteristics: cooperative brood care (including care of offspring from other individuals), overlapping generations within a colony of adults, or a division of labor into reproductive and non-reproductive groups. Eusociality is distinguished from all other social systems because individuals of at least one group usually lose the ability to perform at least one behavior characteristic of individuals in another group. Non-limiting examples of social insects include Hymenoptera (e.g., ants, bees, or wasps) and in Isoptera (e.g., termites).

As used herein, an invertebrate refers to an animals that neither possesses nor develops a vertebral column (commonly known as a backbone or spine), derived from the notochord. This includes all animals apart from the subphylum Vertebrata. Familiar examples of invertebrates include arthropods (insects, arachnids, crustaceans, and myriapods), mollusks (chitons, snail, bivalves, squids, and octopuses), annelid (earthworms and leeches), and cnidarians (hydras, jellyfishes, sea anemones, and corals). In some embodiments, the invertebrate as used herein is an insect. In further embodiments, the invertebrate as used herein is a social insect. In yet further embodiments, the invertebrate as used herein is a pollinator, such as a honey bee.

The term “bee colony” or “honeybee colony” as used herein, refers to a social unit of bees, e.g., honeybees comprising a colony. The social unit can be of any system organization utilized by bees, which has the purpose of facilitating survival of the group or colony. Typically, a “bee colony” consists of several thousand bees that cooperate in nest building, food collection, and brood rearing. Each member of a “bee colony” has a definite task to perform, and it takes the combined efforts of the entire colony to survive and reproduce. A colony typically comprises a single queen, thousands of workers, and hundreds of drones during late spring and summer. Thus, a bee colony is a “population of honeybees”.

Typically, a “honeybee colony” peaks from late spring to summer and reaches a low point in winter. The social structure of the colony is maintained by the queen and workers and depends on an effective system of communication. Domesticated honeybees are cultivated in “beehives” or “honeybee hives.” Thus, the term “beehive” or “honeybee hive” refers to a structure that functions as a habitation for a colony of bees, e.g., a colony of honeybees.

The term “effective amount” or “an amount effective to” or any grammatically equivalent term or expression refers to the amount that, when administered to e.g., a honeybee queen or honeybee colony, for treating a disease or condition, is sufficient to effect treatment for that disease e.g., European foulbrood disease. Typically, an effective dose of antigen for immunizing a queen bee and her respective brood is about 1.5×10⁵ or about 1.5×10⁶ or about 1.5×10⁷ or about 1.5×10⁸ to about 1.5×10¹¹ or 2.2×10¹⁰ antigen units per dose and ranges therebetween. An “effective amount” refers to that amount of a composition which when fed to a queen bee is sufficient to vaccinate the queen and the larvae she produces such that larvae from the vaccinated queen are at least 20%, or alternatively, at least 25% or at least 30%, or alternatively at least 40% more resistant against infection with the vaccine as compared to an unvaccinated queen against disease than are larvae from an unvaccinated queen.

As used herein, “treating” or “treatment” of a disease in an invertebrate refers to (1) preventing the symptoms or disease from occurring in an invertebrate in that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis. In the other aspect, treatment refers to prophylaxis or reducing the incidence of disease in the larvae from a vaccinated queen or other brood bearing insects and other invertebrates.

The term “prophylactic” or “vaccine” refers to an agent that acts to prevent a disease e.g., a honeybee disease, such as e.g., European Foulbrood caused by the bacterium Melissococcus plutonius.

The term “vaccinate” as used herein, refers to means for producing immunity against a disease e.g., producing immunity to Melissococcus plutonius, so as to prevent a disease or condition from occurring (prophylactic treatment) or inhibiting the disease from spreading (slowing or arresting its development).

### I. Modes for Carrying Out the Disclosure

Honeybees, in particular Apis mellifera, are the primary pollinators of most commercial crops in North America, and are the most actively managed pollinators in the world. Accordingly, honeybees have a significant economic impact, and therefore, maintaining healthy bee colonies is an essential aspect of much agricultural practice.

Recently, commercial honeybee colonies have suffered extensive losses due to diseases such as European foulbrood disease caused by the bacterium Melissococcus plutonius.

European Foulbrood (EFB) and AFB are catastrophic diseases of bee colonies which is both contagious and extremely challenging to treat. It can wipe out entire colonies. Often, the most effective treatment for AFB is to simply burn the hive and for EFB the use of antibiotics for the colony and bleach to disinfect the frames and hive. New regulatory advice for handling EFB is simply to burn all hives when antibiotic treatment is ineffective. Fortunately, the present disclosure provides novel methods for vaccinating honeybee queens again European foulbrood and thereby, prophylactically immunizing honeybee colonies to prevent disease.

It has previously been described that immunization of bees can be accomplished by inoculating queen bees with dead, non-disease causing bacteria of the same strain (see e.g., U.S. Patent Application Publication 2018/021529). Applicant is the first to disclose that other non-disease causing bacterial species can be used to prepare a vaccine to prophylactically immunize the bees against European or American foulbrood disease. Applicant is the first to disclose that specific match between the disease pathogen and the vaccine strain is not required. Applicant also is the first to disclose that inoculating queen bees with Melissococcus protects against non-disease causing bacterial and disease-causing bacterial disease

Thus, one aspect of the disclosure is a method of protecting insects from diseases by applying an immunization stimulus derived from a species of non-disease causing bacteria that is in the same genus as the disease pathogen and thereby offering broad-spectrum generalized protection. Non-limiting examples of insects that can be protected using the vaccine formulations disclosed herein include e.g., honeybees (Apis sp.), bumblebees (Bombus sp.) which can be protected from infection and disease caused by e.g., in Apicystis bombi, Crithidia bombi, Paenibacillus larvae the causative agent of American foulbrood, Melissococcus plutonius the causative agent of European foulbrood. As used herein, the term “applying an immunization stimulus” means to prevent disease from disease causing bacterium.

The term “genus” as used herein has its customary meaning as known in the art. In general, genus is defined as taxonomic rank used in the biological classification of living organisms, in the hierarchy of biological classification, genus comes above species and below family. By way of example, the Paenibacillus genus is of facultative anaerobic, endospore-forming bacteria, classified by Ash et al. 1994 (see e.g., Ash, C., Priest, F. G. & Collins, M. D. (1994). Paenibacillus gen. nov. and Paenibacillus polymyxa comb. nov. In Validation of the Publication of New Names and New Combinations Previously Effectively Published Outside the IJSB, List no. 51. Int J Syst Bacteriol 44, 852). Disease pathogen is an infective biological agent, that causes an illness in a host, characterized by certain features such disturbing the function of an organism, and seriously impairs host, including its demise. Broad spectrum generalized protection is achieved when immunization with a bacteria of specific genus or a multiple species of the genus protects against infection of the host then encountering a disease causing pathogen that was not included in vaccine preparation.

In exemplary embodiments, the disclosure provides a novel vaccine formulation and methods for the prevention of disease from disease causing pathogens such as European Foulbrood diseases (EFB) or American Foulbrood diseases (AFB) in honeybees and other insects, via vaccination of queen bees with dead, non-disease causing Melissococcus plutonius or Paenibacillus sp. e.g Paenibacillus larvae, Paenibacillus alvei (PA) and Paenibacillus dentritiformis (PD). In one aspect, the non-disease causing bacterial species comprises whole cells or cell wall fragments of at least one dead, non-disease causing bacterial species. Further, the disclosure relates to generally immunizing insects by using closely related bacteria in addition to using disease causing strains.

In one aspect, provided is a method of preventing an infectious disease caused by a pathogen in an invertebrate (such as a social insect). The method comprises, or essentially consists of, or yet further consists of administering the invertebrate an effective amount of a non-disease causing microorganism or an isolated component thereof wherein the administered microorganism is not the pathogen.

In another aspect, provided is method of preventing an infectious disease caused by a pathogen in an invertebrate (such as a social insect). The method comprises, or essentially consists of, or yet further consists of administering a parent of the invertebrate an effective amount of a non-disease causing microorganism or an isolated component thereof wherein the administered microorganism is not the pathogen. In further embodiments, the microorganism is within the same class of the pathogen. In some embodiments, the microorganism is within the same order of the pathogen. In some embodiments, the microorganism is within the same family of the pathogen. In some embodiments, the microorganism is within the same genus of the pathogen. In some embodiments, the parent is the maternal parent.

In some embodiments, the microorganism is a dead microorganism. In some embodiments, the microorganism comprises a cell wall and the isolated component of the microorganism comprises cell walls. In some embodiments, the microorganism is a bacterium or a fungus.

In some embodiments, the method as disclosed herein comprises, or essentially consists of, or yet further consists of feeding the invertebrate or the parent thereof with the microorganism or an isolated component thereof and an invertebrate food or water.

In some embodiments, the invertebrate is an insect, for example, a social insect. In some embodiments, the invertebrate is selected from a bee, a shrimp, a mealworm, or a grasshopper. In some embodiments, the invertebrate is a honeybee or a honeybee larvae. In further embodiments, the method comprises administering the microorganism or an isolated component thereof to the honeybee, a honeybee queen, a nurse honeybee, or any combination thereof.

In some embodiments, the administration comprises feeding the queen bee wafer of queen bee candy (e.g., from about 1 to 150 gram, or alternatively from about 10 to 125 gram, or alternatively from about 20 to 90 gram, or alternatively from about 25 to about 125 gram, or alternatively from about 50 to about 100 gram, or alternatively about 60, or about 70, or about 80 grams, or about 90 grams, or about 100 gram wafer) of queen candy comprising, or essentially consisting of, or yet further consisting of from about 1.5×10⁷ to about 1.5×10¹¹ antigen units per dose of the microorganism or an isolated component thereof.

In some embodiments, the microorganism comprises, or essentially consists of, or yet further consists of a Paenibacillus bacterium. In further embodiments, the microorganism comprises, or essentially consists of, or yet further consists of a Paenibacillus bacterium selected from the group consisting of Paenibacillus larvae, Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, Paenibacillus xylanisolvens, or any combination thereof. In yet further embodiments, the microorganism comprises, or essentially consists of, or yet further consists of Paenibacillus alvei, Paenibacillus dentritiformis and Paenibacillus larvae. Additionally or alternatively, the pathogen comprises, or essentially consists of, or yet further consists of Melissococcus plutonius and the infectious disease is European Foulbrood Disease (EFB).

In some embodiments, the microorganism comprises, or essentially consists of, or yet further consists of Melissococcus plutonius. Additionally or alternatively, the pathogen comprises, or essentially consists of, or yet further consists of a Paenibacillus bacterium and optionally comprises the microorganism comprises, or essentially consists of, or yet further consists of Melissococcus plutonius. In some embodiments, the pathogen comprises, or essentially consists of, or yet further consists of a Paenibacillus bacterium selected from the group consisting of Paenibacillus larvae, Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, Paenibacillus xylanisolvens, or any combination thereof. In further embodiments, the pathogen comprises, or essentially consists of, or yet further consists of Paenibacillus alvei, Paenibacillus dentritiformis and Paenibacillus larvae. In some embodiments, the infectious disease treated with the vaccine is American Foulbrood Disease (AFB).

In some embodiments, the method as disclosed herein comprises, or essentially consists of, or yet further consists of an administration of about 1.5×10⁵ to about 1.5×10¹¹ or 1×10⁵ to about 2.0×10¹⁰ antigen units of the microorganism or an isolated component thereof per dose.

In yet another aspect, provided is a method or a kit for preparing a composition comprising a microorganism or an isolated component thereof as disclosed herein. In some embodiments, the composition is for uses as disclosed herein. In some embodiments, the method comprises, or essentially consists of, or yet further consists of inactivating or killing the microorganism. In some embodiments, the method comprises, or essentially consists of, or yet further consists of admixing the microorganism or an isolated component thereof with an invertebrate food. In some embodiments, the food is a queen bee wafer.

Also provided is a composition or a kit for uses as disclosed herein. In some embodiments, the composition or kit comprises, or essentially consists of, or further consists of an microorganism or an isolated component thereof and instructions for use.

Unless specified, any embodiment or aspect can be combined or substituted with any other embodiment or aspect as disclosed herein.

### II. Preparation of a Composition for Preventing Bacterial Disease in an Invertebrate Population

### A. General Methods

This disclosure utilizes routine techniques in the field of honeybee husbandry. Basic texts disclosing terms and methods in honeybee husbandry include e.g., Queen Rearing and Bee Breeding Harry H. Laidlaw and Robert E. Page (1997). The below methods are intended to be exemplary only, and can be applied more broadly to other insects, diseases and bacteria.

### B. Preparation of Oral Vaccine

In some embodiments, the disclosure provides compositions comprising organisms of a specific phenotype. The composition protects against diseases caused not only by species in vaccine preparation, but also against similar diseases caused by other species in the same genus. Such species that can protect against the disease caused by another species is called a non-disease organism.

For example, Melissococcus plutonius causes European Foulbrood. Fortunately, the disclosure provides for the ability to protect against Melissococcus plutonius infections as well as against other brood infections. Therefore, the term “disease-causing” and/or “non-disease causing” are relative terms that relates to the targeted disease. For example, with reference to European Foulbrood, wherein the causative agent is Melissococcus plutonius, Melissococcus plutonius is the “disease causing” bacterium. In this context, other agents such as Paenibacillus sp. are “non-disease causing.” In one aspect, the non-disease causing bacterial species comprises whole cells or cell wall fragments of at least one dead non-disease causing bacterial species.

In embodiments, vaccine compositions comprising non-disease organisms are prepared as disclosed herein in Example 1. Briefly, non-disease causing bacteria are grown on agar plates or grown in fermenters and collected by methods known in the art. Typically, the non-disease causing bacteria or other organism is related to a disease causing bacterium, typically of the same genus or family or order or class, but different species.

The collected bacteria are autoclaved or otherwise inactivated. Any method known in the art for killing bacteria may be used. Following killing, the cells are lysed and large fragments of separated by centrifugation for between 1-4 hours (or longer, depending on the volume, size and concentration) at a speed from about 10,000 g to about 60,000 g. The supernatant is recovered and further fractionated as disclosed in Example 1.

The supernatant can be freeze dried, lyophilized or in solution and can be administered to the animals in different forms, such as e.g., feed, spray, injection. In the case of the bees, one mode of administration, includes but is not limited to a preparation of the queen candy, which can be purchased prepacked at beekeeping supply stores or prepared by methods known in the art.

In an exemplary embodiment, the vaccine preparation is given to worker bees that mixes the vaccine with royal jelly in the mandibular glands and feed it to the queen bee or it is given directly to a queen bee to effectively immunize the queen and her resulting brood of larvae against the pathogenic bacterial disease (e.g. against European foulbrood), for example a vaccine preparation with Paenibacillus can protect against European Foulbrood from 3-12 months.

### III. Using Vaccine Compositions

The oral vaccine technology disclosed herein, is administered via the queen bee feed (‘queen candy’), any other bee feed that is taken up by the queen or the worker bees and fed via royal jelly to the queen bee or queen larvae. It is based on the concept of trans-generational immune priming (see e.g., Salmela H, Amdam G V, Freitak D (2015) Transfer of Immunity from Mother to Offspring Is Mediated via Egg-Yolk Protein Vitellogenin. PLOS Pathog 11(7):e1005015. doi.org/10.1371/journal.ppat.1005015.), which immunizes the queen bee, and in turn transfers the antigen and the acquired innate immune signal to the eggs and thus protecting newly hatched larvae and bees against harmful pathogens.

As is known in the art, insect immune systems can recognize specific pathogens and prime offspring immunity. High specificity of immune priming can be achieved when insect females transfer immune elicitors into developing oocytes. The molecular mechanism behind this transfer occurs through the egg-yolk protein vitellogenin. Vitellogenin binds to bacteria or fragments of bacteria which comprise cell wall, and recognizes pathogen-associated molecular patterns to transmit immune-priming signals (see e.g., Salmela H, Amdam G V, Freitak D (2015) supra).

Without being bound by theory it is believed that certain cell wall markers, such as within an bacterial genus are shared across the genus. Exposure to these cell wall markers, either as part of whole cells or cell wall fragments, stimulates immunity to other members of the genus, not just to the species from which the cell wall markers are derived. Thus, exposure to the cell wall markers from one species of the genus e.g., a non-disease causing species, stimulates immunity to other members of the genus, including disease-causing (pathogenic) members of the genus. Thus, in exemplary embodiments, immunizing with compositions comprising at least P. alvei or P. larvae protects against the infection Melissococcus plutonius.

In an embodiment, the composition of oral vaccine is delivered as part of the normal husbandry practice for queen bees. The queens together with 6-10 worker bees are placed into ‘queen cages’ supplied with sufficient feed (queen candy) to last up to one or two weeks. The oral vaccine is added to the queen candy, on which the queen bee and the worker bees are fed for 3-7 days after which she is placed into the new hive, ready to lay eggs and create the new protected bee colony.

In another embodiment the vaccine is places into the queen candy in the queen shipping box or into the nuc or queen rearing hive where nurse bees consume the vaccine with the queen candy and transport it to their royal jelly glands where it is mixed with royal jelly that is fed to the queen bee larvae or the queen bee.

### EXAMPLES

### Example 1

The following Example illustrates an exemplary method for preparing a composition comprising an effective amount of more than one species of dead, fragmented Paenibacillus.

Paenibacillus alvei (PA) and Paenibacillus larvae (PL) (or other antigens) are prepared to provide a vaccine for honeybee queens. The method disclosed below provides for uniformity across all vaccine preparations.

Seed agar plates are innoculated from frozen glycerol stocks of Paenibacillus alvei (PA) or Paenibacillus larvae (PL) or other Paenibacillus sp. The inoculated plates are grown at 35° C. in a darkened growth chamber for 4-12 days.

Paenibacillus colonies are harvested by washing the plates with 5 mL ice-cold H₂O and scraping them off into a falcon tube (or glass bottle). The optical density and 600 nm (OD600) is measured using standard methods. Equal volumes of harvested bacteria culture and H₂O are mixed in two replicates into cuvettes. Based on the OD600 reading, antigen solution was prepared to the desired concentration—at least 1.5×10⁸ bacterial cells/mL. Bacterial solution is autoclaved or 15 min at 121° C. to kill the bacteria.

### Method of Obtaining Bacterial Fragments to be Used as Antigen

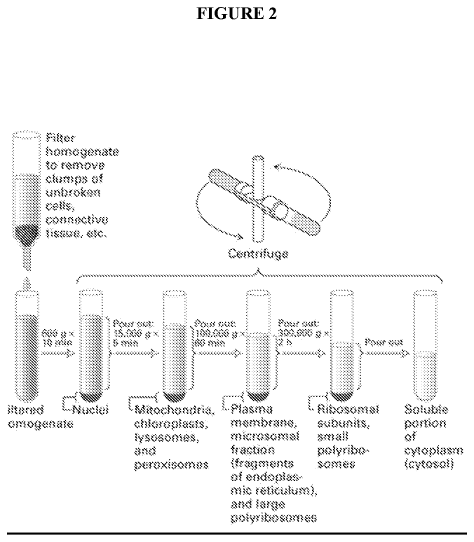

A protein extraction mix sold under BUGBUSTER® can be used on fresh or frozen cell pellets. Cells are harvested from liquid culture by centrifugation at 10,000×g for 10 min using a weighed centrifuge tube. For small scale extractions (1.5 ml or less), centrifugation can be performed in a 1.5-ml tube at 14,000-16,000×g. Liquid is decanted and the pellet tis allowed to drain, removing as much liquid as possible. Pellet is weighed. Once cells have been harvested, but not autoclaved, they can be pelleted and stored at +4° C. The cell pellet is resuspended at room temperature with BUGBUSTER® Master Mix by pipetting or gentle vortexing, using 5 ml reagent per gram of wet cell paste. This typically corresponds to about 2.5 ml per 50-ml culture. The cell suspension is incubated on a shaking platform or rotating mixer at a slow setting for 10-20 min at room temperature. Incubated for 1 h, shaking, +25° C. Insoluble cell debris is removed by centrifugation at 16,000×g for 20 min at 4° C. The supernatant is transferred to a fresh tube. Clarified extracts are maintained on ice for short term storage (2-3 h) or frozen at −20° C. until needed. Cell homogenate is transferred into centrifuge tubes. Centrifugation is done according to the scheme below, supernatant is transferred to new centrifuge tube. See FIG. 2.

Centrifugation steps used (at 4° C.): 1) 10 min 800×g; 2) 10 min 1500×g; 3) 60 min 10000×g; and 4) 3 h 20000×g. Add 500 μl PBS to each pellet and vortex. Collected fragments are used as an antigen in the wafer to vaccinate the queens. See FIG. 2.

### Example 2

The following Example illustrates an exemplary method for immunizing a honeybee hive against American foulbrood disease using non-disease causing Paenibacillus.

One wafer comprising a composition of non-disease causing Paenibacillus is used to vaccinate up to at least 7 queens. The wafer comprises 80 g queen candy (commercial bee feed sold by Nordzucker), 1 mL antigen containing 1.5×10⁸-1.5×10¹⁰ antigen in 1× Phosphate buffered saline (PBS; 1× dilution, pH 6.6-7.2, 137 mM NaCl, 2.7 mM KCl, 9.5 mM Phosphate buffer). The concentration of the antigen is measured photometrically, with as OD600.

The unit for vaccination is the honeybee queen, before placement into a hive or while in the hive. As is well known in the art, to introduce a queen bee into a hive, the queen is first placed alone in a queen cage and fed queen candy. In this Example, about one week prior to being placed in a hive, queens are fed commercial bee feed wafers that comprise the composition of non-disease causing Paenibacillus antigen(s) prepared as disclosed herein in Example 1.

After feeding the queen bee the queen candy wafer comprising the non-disease causing Paenibacillus antigens, the queen bee is placed in the hive.

After placement into a hive, queens start laying eggs within 3-5 days and continue until the end of the brood production period. The larvae laid by the queen are at least 30% more resistant against infection with European foulbrood (Melissococcus plutonius).

Resistance against infection is determined by grafting freshly hatched (12-36 hours) honeybee larvae from both vaccinated and placebo treated hives into the artificial rearing conditions in the lab. All the larvae are then subjected either to oral infection treatment, consisting of food inoculated with living Melissococcus plutonius or are subjected to control treatment (food with no Melissococcus plutonius). The survival is monitored for 8 days and the resistance to infection measured by comparing the survival of immunized vs non immunized larvae.

### Example 3

Two different vaccines were prepared and administered to the queens, as described in WO 2017/017313, filed Feb. 2, 2017 and U.S. Pat. No. 10,994,001, each incorporated herein by reference. Additional methods for the preparation of the vaccine are described herein and illustrated in FIG. 2.

Cross-Protection Provided by AFB Vaccine Against the Infection with EFB.

6 hives in total were used in the study. 3 hives were established by Placebo treated queen and 3 hives were established with AFB-bacterin (AFB Strain 9815, dose per queen 5×10⁷, which was diluted 1:1 in the queen candy) fed queen. 1-day old larvae were collected from the frames, brought to lab and using artificial infection and rearing condition were exposed to control (PBS added to the larval feed) or with 0.09×10⁸ cells of EFB-60 strain. Larvae were kept at relative humidity of 60% at 36 C in constant darkness, and provided with clean Petri dish and new food every second day over 9 day period, the survival was monitored daily. Dead larvae were removed.

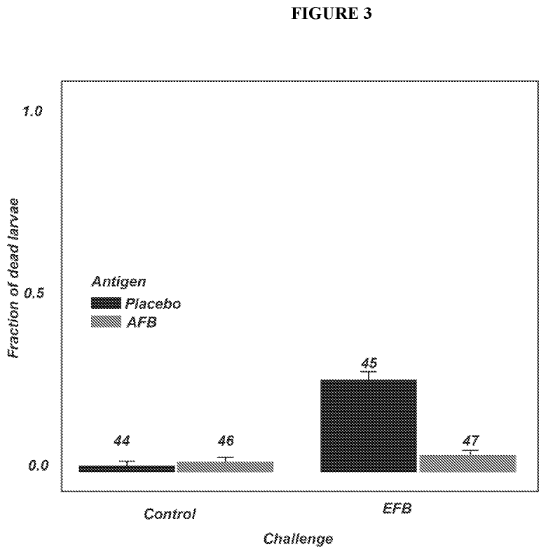

Vaccination had no effect on the larval survival under challenge free condition (X²=1.186, df=1, p=0.276), in the case of the challenge with infectious dose of EFB bacteria, vaccinated larvae were surviving better compared to larvae coming from Placebo treated hives (X²=9.365, df=1, p=0.002). See FIG. 3

Cross-Protection Provided by EFB Vaccine Against the Infection with AFB.

17 hives in total were used in the study. 8 hives were established by Placebo treated queen and 9 hives were established with EFB-bacterin (Strain 119, dose pre queen 2.14×10⁸ in queen candy fed queen. 1-day old larvae were collected from the frames, brought to lab and using artificial infection and rearing condition were exposed to control (PBS added to the larval feed) or with 10.000 cells of AFB strain. Larvae were kept at relative humidity of 60% at 36 C in constant darkness, and provided with clean Petri dish and new food every second day over 9 day period, the survival was monitored daily. Dead larvae were removed.

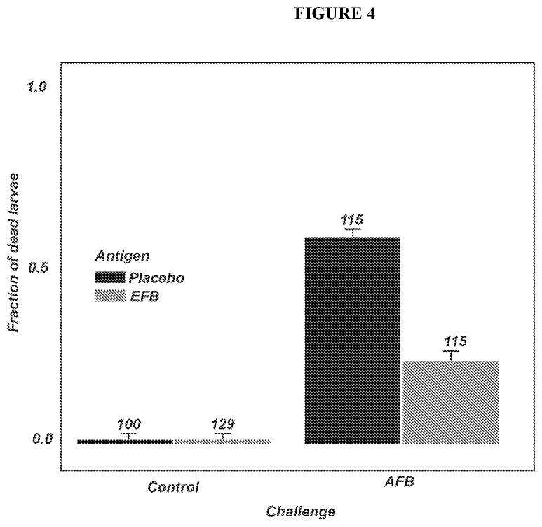

Vaccination had no effect on the larval survival under challenge free condition (X²=0.119, df=1, p=0.730), in the case of the challenge with infectious dose of AFB bacteria, vaccinated larvae were surviving better compared to larvae coming from Placebo treated hives (X²=6.188, df=1, p=0.013). See FIG. 4.

### EQUIVALENTS

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

1. A method for immunizing a queen bee or passing immunity from the bee queen to bee larvae produced by the queen bee against a bacterial disease, comprising administering a vaccine composition comprising an effective amount of dead non-disease whole cells or cell wall fragments of a non-disease causing bacterial species to the bee queen thereby immunizing the queen bee and the bee larvae produced by the queen bee.

2. The method of claim 1, wherein the queen bee is a honeybee queen bee and the bee larvae are honeybee larvae produced by the honeybee queen bee.

3. The method of claim 1 wherein the bacterial disease is caused by Enterococcus, optionally Melissococcus sp. and the dead non-disease causing bacterial species comprises Paenibacillus sp.

4. The method of claim 1 wherein the bacterial disease is caused by Paenibacillus sp. and the dead non-disease causing bacterial species comprises Melissococcus sp.

5. The method of any of claims 1-4, claim 1, wherein the vaccine composition further comprises whole cells or cell wall fragments of at least one dead bacterial species of selected from Melissococcus plutonius, Paenibacillus, Paenibacillus larvae, Paenibacillus alvei Paenibacillus dentritiformis, or a combination thereof.

6. The composition of claim 3, wherein the dead, non-disease causing bacterial species is selected from the group consisting of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, Paenibacillus xylanisolvens, or a combination thereof.

7. The method of claim 4, wherein the dead, non-disease causing bacterial species comprises Melissococcus plutonius.

8. The method of claim 1, wherein the vaccine composition comprises from about 1.5×10⁵ to about 2.0×10¹⁰ antigen units of whole cells or cell wall fragments per dose.

9. A method of immunizing a bee larvae against Foulbrood disease, comprising administering an effective amount of a composition comprising a dead non-disease causing bacteria to a worker bee or a bee queen, thereby immunizing the bee larvae produced by the queen bee.

10. The method of claim 9, wherein the queen bee is a honeybee queen.

11. (canceled)

12. The method of claim 9, wherein the Foulbrood disease is European Foulbrood caused by Melissococcus plutonius and the dead non-disease causing bacteria comprises Paenibacillus sp.

13. The method of claim 9, wherein the Foulbrood disease is American Foulbrood and is caused by Paenibacillus sp and the dead non-disease causing bacteria comprises Melissococcus sp.

14. (canceled)

15. The method of claim 12, wherein the dead non-disease species of Paenibacillus is selected from the group of Paenibacillus alvei, Paenibacillus dentritiformis, Paenibacillus amylolyticus, Paenibacillus campinasensis, Paenibacillus chondroitinus, Paenibacillus chungangensis, Paenibacillus doosanensis, Paenibacillus glucanolyticus, Paenibacillus humicus, Paenibacillus lactis, Paenibacillus lautus, Paenibacillus lentimorbus, Paenibacillus maceran, Paenibacillus macerans-like, Paenibacillus macquariensis, Paenibacillus motobuensis, Paenibacillus pabuli, Paenibacillus phoenicis, Paenibacillus polymyxa, Paenibacillus popilliae, Paenibacillus puldeungensis, Paenibacillus residui, Paenibacillus stellife, Paenibacillus thiaminolyticus, Paenibacillus validus, Paenibacillus xylanisolvens, or a combination thereof.

16.-18. (canceled)

19. The method of claim 13, wherein the dead non-disease species is Melissococcus plutonius.

20. The method of claim 1, wherein the administering comprises feeding the queen bee a wafer of queen candy comprising from about 1.5×10⁵ to about 2.0×10¹⁰ antigen units per dose of at least one dead non-disease species or fragments thereof.

21. A method for preparing a vaccine for an insect or other invertebrate, the method comprising isolating whole cells or cell wall fragments from at least one dead of a bacterial Melissococcus plutonius.

22. The method of claim 21, further comprising admixing the isolated antigen units with an insect or other invertebrate food, such as a queen bee wafer.

23. The method of claim 21, further comprising formulating the isolated whole cells or cell wall fragments to provide from about 1.5×10⁵ to about 2.0×10¹⁰ antigen units per dose.

24. A vaccine composition prepared by a method of claim 21.

25. A kit comprising the vaccine composition of claim 24, and instructions for use.
